(19) United States
(12) Patent Application Publication
(10) Pub. No.: US 2025/0152943 A1
(43) Pub. Date: May 15, 2025

(54) Closed Loop DBS Using Evoked Potentials

(71) Applicant: Boston Scientific Neuromodulation Corporation, Valencia, CA (US)

(72) Inventors: Tianhe Zhang, Studio City, CA (US); Rosana Esteller, Santa Clarita, CA (US)

(21) Appl. No.: 18/937,888

(22) Filed: Nov. 5, 2024

Related U.S. Application Data
(60) Provisional application No. 63/597,604, filed on Nov. 9, 2023.

(51) Int. Cl. A61N 1/36; A61N 1/372

(52) U.S. Cl. CPC A61N 1/36139; A61N 1/3615; A61N 1/36171; A61N 1/36175; A61N 1/36185; A61N 1/37241

(57) ABSTRACT

Methods and systems for optimizing and/or maintaining stimulation during deep brain stimulation (DBS) are described. The methods and systems involve determining evoked neural responses, namely evoked resonant neural activity (ERNA) evoked in a patient's brain by the stimulation. ERNA features corresponding to the patient's brain state during the absence and during the presence of therapeutic stimulation are determined. The ERNA features are used to guide and to maintain stimulation parameters for therapeutic stimulation.

### CROSS REFERENCE TO RELATED APPLICATIONS

This is a non-provisional application of U.S. Provisional Patent Application Ser. No. 63/597,604, filed Nov. 9, 2023, which is incorporated herein by reference in its entirety, and to which priority is claimed.

### FIELD OF THE INVENTION

This application relates to deep brain stimulation (DBS), and more particularly, to methods and systems for using sensed neural responses for facilitating aspects of DBS.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) context. DBS has been applied therapeutically for the treatment of neurological disorders, including Parkinson's Disease, essential tremor, dystonia, and epilepsy, to name but a few. Further details discussing the treatment of diseases using DBS are disclosed in U.S. Pat. Nos. 6,845,267, and 6,950,707. However, the present invention may find applicability with any implantable neurostimulator device system.

Each of these neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise a handheld external control device to remotely instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects. For example, bilateral DBS of the subthalamic nucleus has been shown to provide effective therapy for improving the major motor signs of advanced Parkinson's disease, and although the bilateral stimulation of the subthalamic nucleus is considered safe, an emerging concern is the potential negative consequences that it may have on cognitive functioning and overall quality of life (see A. M. M. Frankemolle, et al., Reversing Cognitive-Motor Impairments in Parkinson's Disease Patients Using a Computational Modelling Approach to Deep Brain Stimulation Programming, Brain 2010; pp. 1-16). In large part, this phenomenon is due to the small size of the subthalamic nucleus. Even with the electrodes located predominately within the sensorimotor territory, the electrical field generated by DBS is non-discriminately applied to all neural elements surrounding the electrodes, thereby resulting in the spread of current to neural elements affecting cognition. As a result, diminished cognitive function during stimulation of the subthalamic nucleus may occur do to non-selective activation of non-motor pathways within or around the subthalamic nucleus.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads but are also distributed circumferentially around the neurostimulation leads as segmented electrodes, can be used.

To facilitate such selection, the clinician generally programs the external control device, and if applicable the neurostimulator, through a computerized programming system. This programming system can be a self-contained hardware/software system or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters.

When electrical leads are implanted within the patient, the computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that best addresses the neurological disorder(s).

In the context of DBS, the brain is dynamic (e.g., due to disease progression, motor re-learning, or other changes), and a program (i.e., a set of stimulation parameters) that is useful for a period of time may not maintain its effectiveness and/or the expectations of the patient may increase. Further, physicians typically treat the patient with stimulation and medication, and proper amounts of each are required for optimal therapy. In particular, a patient's stimulation needs may be impacted by their medication state. Additionally, the need for stimulation and/or medication may fluctuate across the day and week, depending on activities of daily living, especially sleep and activity.

Thus, there is a need for closed loop feedback that can be used to adjust stimulation parameters as the patient's stimulation needs change with time or based on their medication state.

### SUMMARY

Disclosed herein are systems for providing deep brain stimulation (DBS) to a patient's brain using one or more electrode leads implanted in the patient's brain, wherein each of the one or more electrode leads comprises a plurality of electrodes configured to contact the patient's brain tissue, the system comprising: control circuitry configured to execute a method comprising: using one or more of the electrodes to provide non-therapeutic stimulation to the patient's brain, wherein the non-therapeutic stimulation is configured to evoke first evoked neural resonant activity (ERNA) in the patient's brain and is not configured to treat the patient's symptoms, using one or more of the electrodes to record first signals in the patient's brain indicative of the first ERNA, wherein the first ERNA is indicative of the patient's brain state in the absence of therapeutic stimulation, using one of more of the electrodes to provide therapeutic stimulation to the patient's brain, wherein the therapeutic stimulation is configured to treat the patient's symptoms, using one or more of the electrodes to record second signals indicative of second ERNA, wherein the second ERNA is indicative of the patient's brain state in the presence of therapeutic stimulation, comparing the first ERNA and the second ERNA, and adjusting the therapeutic stimulation based on the comparison. According to some embodiments, the non-therapeutic stimulation has a frequency of about 5 to about 50 Hz. According to some embodiments, the therapeutic stimulation has a frequency of about 100 to about 150 Hz. According to some embodiments, comparing the first and second ERNA comprises extracting one or more features from the first and second signals. According to some embodiments, the one or more features comprise one or more of a latency of one or more peaks and an amplitude of one or more peaks. According to some embodiments, comparing the first and second ERNA comprises measuring a difference between the one or more extracted features of the first signal and the one or more extracted features of the second signal. According to some embodiments, comparing the first and second ERNA comprises determining a ratio of the one or more extracted features of the first signal and the one or more extracted features of the second signal. According to some embodiments, the second signals are recorded while the therapeutic stimulation is provided to the patient's brain. According to some embodiments, recording the second signals comprises: providing the therapeutic stimulation for a first duration, ceasing the therapeutic stimulation, and providing the non-therapeutic stimulation, and recording the second signals in response to the non-therapeutic stimulation. According to some embodiments, the method further comprises providing the non-therapeutic stimulation for a second duration and recording a progression of the ERNA during the second duration. According to some embodiments, adjusting the therapeutic stimulation comprises adjusting the stimulation to maintain a feature of the second ERNA with respect to a threshold value and/or a range of values. According to some embodiments, the method further comprises issuing an alert if the feature of the second ERNA is outside the threshold and/or range of values. According to some embodiments, configuring the therapeutic stimulation comprises determining an optimized electrode configuration for providing the therapeutic stimulation, wherein the optimized electrode configuration comprises optimized one or more electrodes used to deliver the therapeutic stimulation. According to some embodiments, determining an optimized electrode configuration comprises: (i) applying the non-therapeutic stimulation and the therapeutic stimulation using a trial electrode configuration comprising a trial one or more electrodes for delivering the stimulation, (ii) determining if one or more features of the first and second ERNAs differ by greater than a predetermined threshold value, (iii) if the one or more of the features differ by greater than the predetermined threshold value, using the trial electrode configuration as the optimized electrode configuration for the therapeutic stimulation, and (iv) if the one or more of the features do not differ by greater than the predetermined threshold value, iteratively repeating steps (i-iii) with different trial electrode configurations until the one or more of the features differ by greater than the predetermined threshold value for that trial electrode configuration and using that trial electrode configuration as the optimized electrode configuration for the therapeutic stimulation. According to some embodiments, the one or more features comprise one or more of a latency of one or more peaks and an amplitude of one or more peaks.

Also disclosed herein are methods for providing deep brain stimulation (DBS) to a patient's brain using one or more electrode leads implanted in the patient's brain, wherein each of the one or more electrode leads comprises a plurality of electrodes configured to contact the patient's brain tissue, the method comprising: using one or more of the electrodes to provide non-therapeutic stimulation to the patient's brain, wherein the non-therapeutic stimulation is configured to evoke first evoked neural resonant activity (ERNA) in the patient's brain and is not configured to treat the patient's symptoms, using one or more of the electrodes to record first signals in the patient's brain indicative of the first ERNA, wherein the first ERNA is indicative of the patient's brain state in the absence of therapeutic stimulation, using one of more of the electrodes to provide therapeutic stimulation to the patient's brain, wherein the therapeutic stimulation is configured to treat the patient's symptoms, using one or more of the electrodes to record second signals indicative of second ERNA, wherein the second ERNA is indicative of the patient's brain state in the presence of therapeutic stimulation, comparing the first ERNA and the second ERNA, and configuring the therapeutic stimulation based on the comparison. According to some embodiments, the non-therapeutic stimulation has a frequency of about 5 to about 50 Hz. According to some embodiments, the therapeutic stimulation has a frequency of about 100 to about 150 Hz. According to some embodiments, comparing the first and second ERNA comprises extracting one or more features from the first and second signals. According to some embodiments, the one or more features comprise one or more of a latency of one or more peaks and an amplitude of one or more peaks. According to some embodiments, comparing the first and second ERNA comprises measuring a difference between the one or more extracted features of the first signal and the one or more extracted features of the second signal. According to some embodiments, comparing the first and second ERNA comprises determining a ratio of the one or more extracted features of the first signal and the one or more extracted features of the second signal. According to some embodiments, the second signals are recorded while the therapeutic stimulation is provided to the patient's brain. According to some embodiments, recording the second signals comprises: providing the therapeutic stimulation for a first duration, ceasing the therapeutic stimulation and providing the non-therapeutic stimulation, and recording the second signals in response to the non-therapeutic stimulation. According to some embodiments, the method further comprises providing the non-therapeutic stimulation for a second duration and recording a progression of the ERNA during the second duration. According to some embodiments, configuring the therapeutic stimulation comprises adjusting the stimulation to maintain a feature of the second ERNA with respect to a threshold value and/or a range of values. According to some embodiments, the method further comprises issuing an alert if the feature of the second ERNA is outside the threshold and/or range of values. According to some embodiments, configuring the therapeutic stimulation comprises adjusting an amplitude of the stimulation. According to some embodiments, configuring the therapeutic stimulation comprises determining an optimized electrode configuration for providing the therapeutic stimulation, wherein the optimized electrode configuration comprises optimized one or more electrodes used to deliver the therapeutic stimulation. According to some embodiments, determining an optimized electrode configuration comprises: (i) applying the non-therapeutic stimulation and the therapeutic stimulation using a trial electrode configuration comprising a trial one or more electrodes for delivering the stimulation, (ii) determining if one or more features of the first and second ERNAs differ by greater than a predetermined threshold value, (iii) if the one or more of the features differ by greater than the predetermined threshold value, using the trial electrode configuration as the optimized electrode configuration for the therapeutic stimulation, and (iv) if the one or more of the features do not differ by greater than the predetermined threshold value, iteratively repeating steps (i-iii) with different trial electrode configurations until the one or more of the features differ by greater than the predetermined threshold value for that trial electrode configuration and using that trial electrode configuration as the optimized electrode configuration for the therapeutic stimulation. According to some embodiments, the one or more features comprise one or more of a latency of one or more peaks and an amplitude of one or more peaks. According to some embodiments, the method further comprises tracking one or more values of one or more features of the first ERNA over time. According to some embodiments, the method further comprises determining one or more trends of the one or more values of one or more features of the first ERNA over time. According to some embodiments, the one or more trends are indicative of a progression or an improvement in the patient's disease state. According to some embodiments, the method further comprises adjusting the stimulation based on the one or more trends.

The invention may also reside in the form of a programed external device (via its control circuitry) for carrying out the above methods, a programmed implantable pulse generator (IPG) or external trial stimulator (ETS) (via its control circuitry) for carrying out the above methods, a system including a programmed external device and IPG or ETS for carrying out the above methods, or as a computer-readable media for carrying out the above methods stored in an external device or IPG or ETS. The invention may also reside in one or more non-transitory computer-readable media comprising instructions, which when executed by a processor of a machine configure the machine to perform any of the above methods.

### BRIEF DESCRIPTION OF THE DRAWINGS

FIG. 1A shows an Implantable Pulse Generator (IPG).

FIG. 1B shows an electrode lead having split-ring electrodes.

FIGS. 2A and 2B show an example of stimulation pulses (waveforms) producible by the IPG or by an External Trial Stimulator (ETS).

FIG. 3 shows an example of stimulation circuitry useable in the IPG or ETS.

FIG. 4 shows an ETS environment useable to provide stimulation before implantation of an IPG.

FIG. 5 shows various external devices capable of communicating with and programming stimulation in an IPG or ETS.

FIG. 6 illustrates sensing circuitry useable in an IPG.

FIG. 7 illustrates an embodiment of a user interface (UI) for programming stimulation.

FIGS. 8A and 8B illustrate evoked resonant neural activity (ERNA).

FIG. 9 illustrates a workflow for using ERNA to optimize and adjust stimulation parameters.

FIG. 10 illustrates waveforms for evoking ERNA in the absence and in the presence of therapeutic stimulation.

FIG. 11 shows a closed loop feedback control algorithm.

### DETAILED DESCRIPTION

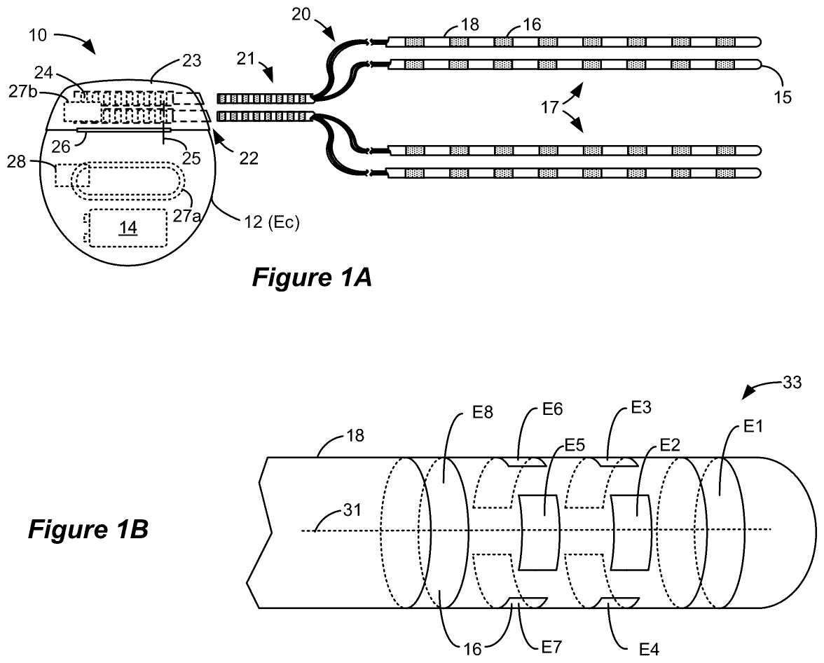

A DBS or SCS system typically includes an Implantable Pulse Generator (IPG) 10 shown in FIG. 1A. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more electrode leads 15 can be used having ring-shaped electrodes 16 carried on a flexible body 18.

In yet another example shown in FIG. 1B, an electrode lead 33 can include one or more split-ring electrodes. In this example, eight electrodes 16 (E1-E8) are shown. Electrode E1 at the distal end of the lead and electrode E8 at a proximal end of the lead comprise ring electrodes spanning 360 degrees around a central axis of the lead 33. In some embodiments, the electrode E1 may be a “bullet tip” electrode, meaning that it can cover the tip of the electrode lead. Electrodes E2, E3, and E4 comprise split-ring electrodes, each of which are located at the same longitudinal position along the central axis 31, but with each spanning less than 360 degrees around the axis. For example, each of electrodes E2, E3, and E4 may span 90 degrees around the axis 31, with each being separated from the others by gaps of 30 degrees. Electrodes E5, E6, and E7 also comprise split-ring electrodes, but are located at a different longitudinal position along the central axis 31 than are split ring electrodes E4, E2, and E3. As shown, the split-ring electrodes E2-E4 and E5-E7 may be located at longitudinal positions along the axis 31 between ring electrodes E1 and E8. However, this is just one example of a lead 33 having split-ring electrodes. In other designs, all electrodes can be split-ring, or there could be different numbers of split-ring electrodes at each longitudinal position (i.e., more or less than three), or the ring and split-ring electrodes could occur at different or random longitudinal positions, etc.

Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 15, and thus the header 23 may include a 2×2 array of eight-electrode lead connectors 22. However, the type and number of leads, and the number of electrodes, in an IPG is application-specific and therefore can vary. The conductive case 12 can also comprise an electrode (Ec).

In a DBS application, as is useful in the treatment of tremor in Parkinson's disease for example, the IPG 10 is typically implanted under the patient's clavicle (collarbone). Lead wires 20 are tunneled through the neck and the scalp and the electrode leads 15 (or 33) are implanted through holes drilled in the skull and positioned for example in the subthalamic nucleus (STN) and the pedunculopontine nucleus (PPN) in each brain hemisphere.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Bluetooth Low Energy (BLE), as described in U.S. Patent Publication 2019/0209851, Zigbee, WiFi, MICS, and the like.

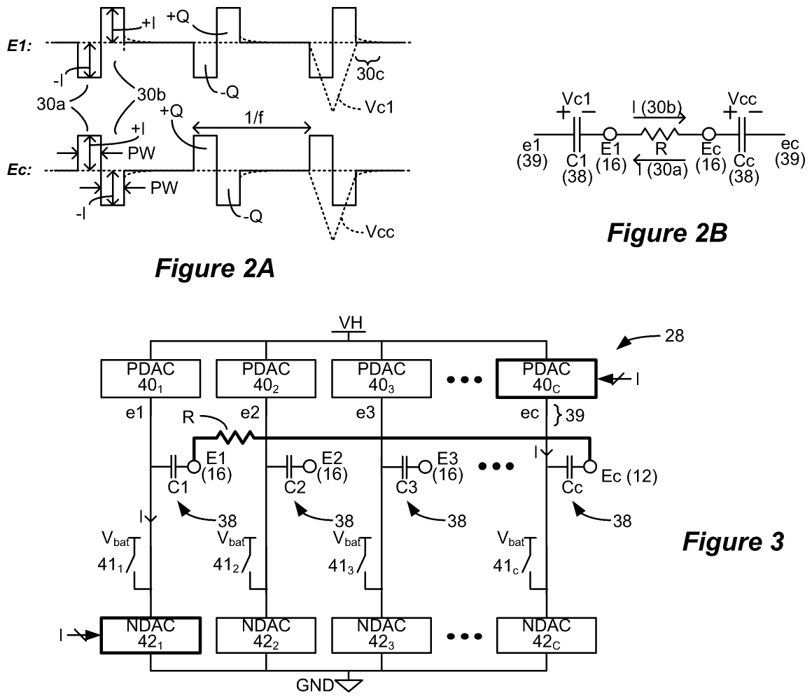

Stimulation in IPG 10 is typically provided by pulses each of which may include a number of phases such as 30a and 30b, as shown in the example of FIG. 2A. In the example shown, such stimulation is monopolar, meaning that a current is provided between at least one selected lead-based electrode (e.g., E1) and the case electrode Ec 12. Stimulation parameters typically include amplitude (current I, although a voltage amplitude V can also be used); frequency (f); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a) to ensure that the net current injected into the tissue is zero. The polarity of the currents at these electrodes can be changed: Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40ᵢ and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node Ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current through a patient's tissue, R, hopefully with good therapeutic effect. In the example shown, and consistent with the first pulse phase 30a of FIG. 2A, electrode E1 has been selected as a cathode electrode to sink current from the tissue R and case electrode Ec has been selected as an anode electrode to source current to the tissue R. Thus PDAC 40_{C} and NDAC 42₁ are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40ᵢ and the electrode nodes ei 39, and between the one or more NDACs 42ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796. The stimulation circuitries described herein provide multiple independent current control (MICC) (or multiple independent voltage control) to guide the estimate of current fractionalization among multiple electrodes and estimate a total amplitude that provide a desired strength. In other words, the total anodic (or cathodic) current can be split among two or more electrodes and/or the total cathodic current can be split among two or more electrodes, allowing the stimulation location and resulting field shapes to be adjusted. For example, a “virtual electrode” may be created at a position between two physical electrodes by fractionating current between the two electrodes.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes eᵢ 39 and the electrodes Eᵢ 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blocking capacitors C1 and Cc associated with the electrodes E1 and Ec used to produce the current, giving rise to voltages Vc1 and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors C1 and Cc is actively recovered, and thus voltages Vc1 and Vcc increase and return to 0V at the end of the second pulse phase 30b.

To recover all charge by the end of the second pulse phase 30b of each pulse (Vc1=Vcc=0V), the first and second phases 30a and 30b are charged balanced at each electrode, with the first pulse phase 30a providing a charge of −Q (−I*PW) and the second pulse phase 30b providing a charge of +Q (+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude (|I|) for each of the opposite-polarity pulse phases 30a and 30b. However, the pulse phases 30a and 30b may also be charged balanced at each electrode if the product of the amplitude and pulse widths of the two phases 30a and 30b are equal, or if the area under each of the phases is equal, as is known.

FIG. 3 shows that stimulation circuitry 28 can include passive recovery switches 41ᵢ, which are described further in U.S. Patent Application Publications 2018/0071527 and 2018/0140831. Passive recovery switches 41ᵢ may be attached to each of the electrode nodes ei 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41ᵢ. As shown in FIG. 3, the other end of the switches 41ᵢ not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery is less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

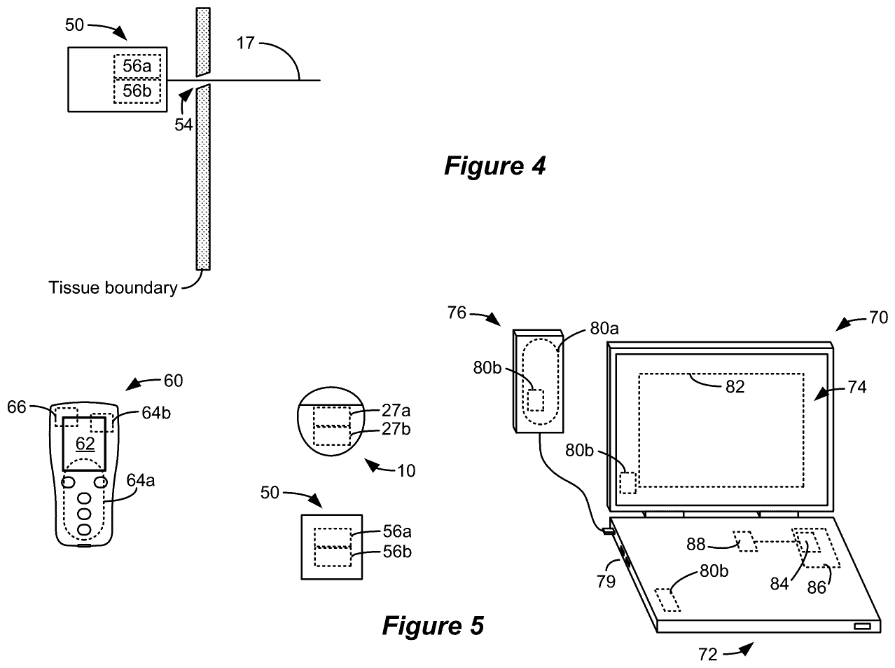

FIG. 4 shows an external trial stimulation environment that may precede implantation of an IPG 10 in a patient, for example, during the operating room to test stimulation and confirm the lead position. During external trial stimulation, stimulation can be tried on the implant patient to evaluate side-effect thresholds and confirm that the lead is not too close to structures that cause side effects. Like the IPG 10, the external trial stimulator (ETS) 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power. The sensing capabilities described herein with regard to the IPG 10, may also be included in the ETS 50 for the purposes described below. As the IPG may include a case electrode, an ETS may provide one or more connections to establish similar returns; for example, using patch electrodes. Likewise, the ETS may communicate with the clinician programmer (CP) so that the CP can process the data as described below.

FIG. 5 shows various external devices that can wirelessly communicate data with the IPG 10 or ETS 50, including a patient hand-held external controller 60, and a clinician programmer (CP) 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

External controller 60 can be as described in U.S. Patent Application Publication 2015/0080982 for example and may comprise a controller dedicated to work with the IPG 10 or ETS 50. External controller 60 may also comprise a general-purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 82 provided on the display 74 of the computing device 72. As one skilled in the art understands, the GUI 82 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 82, can also enable communications via antennas 80a or 80b to communicate stimulation parameters chosen through the GUI 82 to the patient's IPG 10.

The user interface of the external controller 60 may provide similar functionality because the external controller 60 can include similar hardware and software programming as the clinician programmer. For example, the external controller 60 includes control circuitry 66 similar to the control circuitry 88 in the clinician programmer 70 and may similarly be programmed with external controller software stored in device memory.

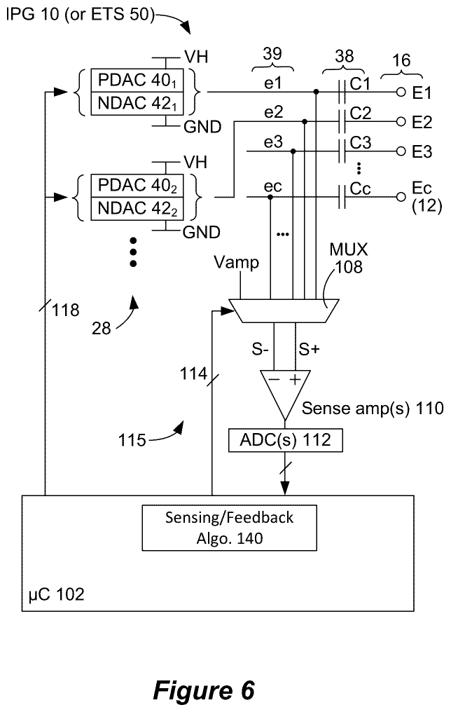

An increasingly interesting development in pulse generator systems is the addition of sensing capability to complement the stimulation that such systems provide. FIG. 6 shows an IPG 10 that includes stimulation and sensing functionality. (An ETS as described earlier could also include stimulation and sensing capabilities). FIG. 6 shows further details of the circuitry in an IPG 10 (and/or ETS) that can provide stimulation and sensing innate or evoked signals. The IPG 10 includes control circuitry 102, which may comprise a microcontroller, such as Part Number MSP430, manufactured by Texas Instruments, Inc., which is described in data sheets at http://www.ti.com/microcontrollers/msp430-ultra-low-power-mcus/overview.html, which are incorporated herein by reference. Other types of controller circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs), such as those described and incorporated earlier. The control circuitry 102 may be configured with one or more sensing/feedback algorithms 140 that are configured to cause the IPG/ETS to sense neural signals and make certain adjustments and/or take certain actions based on the sensed neural signals. The sensing/feedback control algorithms may be configured within memory of the IPG.

The IPG 10 also includes stimulation circuitry 28 to produce stimulation at the electrodes 16, which may comprise the stimulation circuitry 28 shown earlier (FIG. 3). A bus 118 provides digital control signals from the control circuitry 102 to one or more PDACs 40ᵢ or NDACs 42ᵢ to produce currents or voltages of prescribed amplitudes (I) for the stimulation pulses, and with the correct timing (PW, F) at selected electrodes. As noted earlier, the DACs can be powered between a compliance voltage VH and ground. As also noted earlier, but not shown in FIG. 4, switch matrices could intervene between the PDACs and the electrode nodes 39, and between the NDACs and the electrode nodes 39, to route their outputs to one or more of the electrodes, including the conductive case electrode 12 (Ec). Control signals for switch matrices, if present, may also be carried by bus 118. Notice that the current paths to the electrodes 16 include the DC-blocking capacitors 38 described earlier, which provide safety by preventing the inadvertent supply of DC current to an electrode and to a patient's tissue. Passive recovery switches 41ᵢ (FIG. 3) could also be present but are not shown in FIG. 6 for simplicity.

IPG 10 also includes sensing circuitry 115, and one or more of the electrodes 16 can be used to sense innate or evoked electrical signals, e.g., biopotentials from the patient's tissue. In this regard, each electrode node 39 can further be coupled to a sense amp circuit 110. Under control by bus 114, a multiplexer 108 can select one or more electrodes to operate as sensing electrodes (S+, S−) by coupling the electrode(s) to the sense amps circuit 110 at a given time, as explained further below. Although only one multiplexer 108 and sense amp circuit 110 are shown in FIG. 6, there could be more than one. For example, there can be four multiplexer 108/sense amp circuit 110 pairs each operable within one of four timing channels supported by the IPG 10 to provide stimulation. The sensed signals output by the sense amp circuitry are preferably converted to digital signals by one or more Analog-to-Digital converters (ADC(s)) 112, which may sample the output of the sense amp circuit 110 at 50 kHz for example. The ADC(s) 112 may also reside within the control circuitry 102, particularly if the control circuitry 102 has A/D inputs. Multiplexer 108 can also provide a fixed reference voltage, Vamp, to the sense amp circuit 110, as is useful in a single-ended sensing mode (i.e., to set S− to Vamp).

So as not to bypass the safety provided by the DC-blocking capacitors 38, the inputs to the sense amp circuitry 110 are preferably taken from the electrode nodes 39. However, the DC-blocking capacitors 38 will pass AC signal components (while blocking DC components), and thus AC components within the signals being sensed will still readily be sensed by the sense amp circuitry 110. In other examples, signals may be sensed directly at the electrodes 16 without passage through intervening capacitors 38.

According to some embodiments, it may be preferred to sense signals differentially, and in this regard, the sense amp circuitry 110 comprises a differential amplifier receiving the sensed signal S+ (e.g., E3) at its non-inverting input and the sensing reference S− (e.g., E1) at its inverting input. As one skilled in the art understands, the differential amplifier will subtract S− from S+ at its output, and so will cancel out any common mode voltage from both inputs. This can be useful for example when sensing various neural signals, as it may be useful to subtract the relatively large-scale stimulation artifact from the measurement (as much as possible).

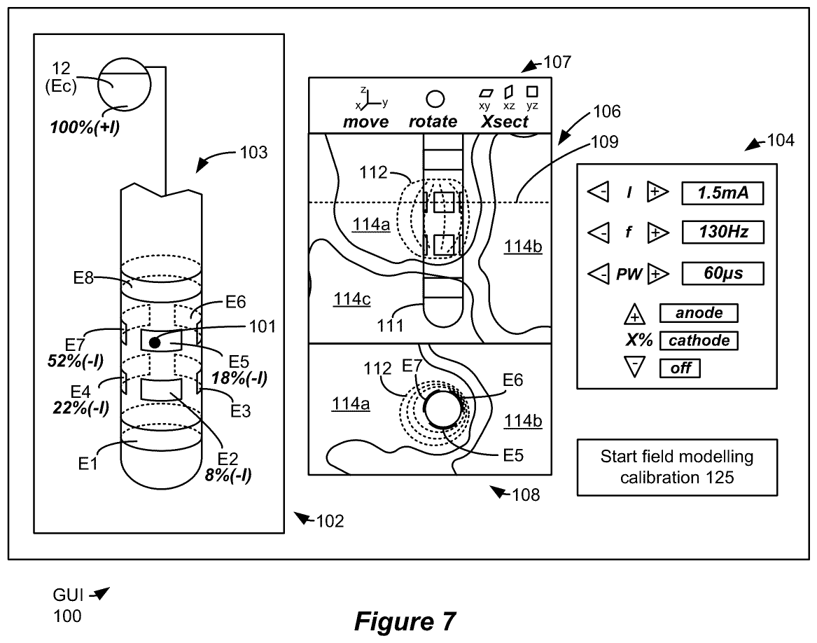

Particularly in the DBS context, it can be useful to provide a clinician with a visual indication of how stimulation selected for a patient will interact with the tissue in which the electrodes are implanted. This is illustrated in FIG. 7, which shows a Graphical User Interface (GUI) 100 operable on an external device capable of communicating with an IPG 10 or ETS 50. Typically, and as assumed in the description that follows, GUI 100 would be rendered on a clinician programmer 70 (FIG. 5), which may be used during surgical implantation of the leads, or after implantation when a therapeutically useful stimulation program is being chosen for a patient. However, GUI 100 could be rendered on a patient external programmer 60 (FIG. 5) or any other external device capable of communicating with the IPG 10 or ETS 50.

GUI 100 allows a clinician (or patient) to select the stimulation program that the IPG 110 or ETS 50 will provide and provides options that control sensing of innate or evoked responses, as described below. In this regard, the GUI 100 may include a stimulation parameter interface 104 where various aspects of the stimulation program can be selected or adjusted. For example, interface 104 allows a user to select the amplitude (e.g., a current I) for stimulation; the frequency (f) of stimulation pulses; and the pulse width (PW) of the stimulation pulses. Stimulation parameter interface 104 can be significantly more complicated, particularly if the IPG 10 or ETS 50 supports the provision of stimulation that is more complicated than a repeating sequence of pulses. See, e.g., U.S. Patent Application Publication 2018/0071513. Nonetheless, interface 104 is simply shown for simplicity in FIG. 7 as allowing only for amplitude, frequency, and pulse width adjustment. Stimulation parameter interface 104 may include inputs to allow a user to select whether stimulation will be provided using biphasic (FIG. 2A) or monophasic pulses, and to select whether passive charge recovery will be used, although again these details aren't shown for simplicity.

Stimulation parameter interface 104 may further allow a user to select the active electrodes—i.e., the electrodes that will receive the prescribed pulses. Selection of the active electrodes can occur in conjunction with a leads interface 102, which can include an image 103 of the one or more leads that have been implanted in the patient. Although not shown, the leads interface 102 can include a selection to access a library of relevant images 103 of the types of leads that may be implanted in different patients.

In the example shown in FIG. 7, the leads interface 102 shows an image 103 of a single split-ring lead 33 like that described earlier with respect to FIG. 1B. The leads interface 102 can include a cursor 101 that the user can move (e.g., using a mouse connected to the clinician programmer 70) to select an illustrated electrode 16 (e.g., E1-E8, or the case electrode Ec). Once an electrode has been selected, the stimulation parameter interface 104 can be used to designate the selected electrode as an anode that will source current to the tissue, or as a cathode that will sink current from the tissue. Further, the stimulation parameter interface 104 allows the amount of the total anodic or cathodic current +I or −I that each selected electrode will receive to be specified in terms of a percentage, X. For example, in FIG. 7, the case electrode 12 Ec is specified to receive X=100% of the current I as an anodic current +I. The corresponding cathodic current −I is split between electrodes E5 (0.18*−I), E7 (0.52*−I), E2 (0.08*−I), and E4 (0.22*−I). Thus, two or more electrodes can be chosen to act as anodes or cathodes at a given time using MICC (as described above), allowing the electric field in the tissue to be shaped. The currents so specified at the selected electrodes can be those provided during a first pulse phase (if biphasic pulses are used), or during an only pulse phase (if monophasic pulses are used).

GUI 100 can further include a visualization interface 106 that can allow a user to view an indication of the effects of stimulation, such as electric field image 112 formed on the one or more leads given the selected stimulation parameters. The electric field image 112 is formed by field modelling, for example, in the clinician programmer 70. Such stimulation field models (SFMs) (also referred to as “volume of tissue active (VTA)) are known in the art. Only one lead is shown in the visualization interface 106 for simplicity, although again a given patient might be implanted with more than one lead. Visualization interface 106 provides an image 111 of the lead(s) which may be three-dimensional.

The visualization interface 106 preferably, but not necessarily, further includes tissue imaging information 114 taken from the patient, represented as three different tissue structures 114a, 114b and 114c in FIG. 7 for the patient in question, which tissue structures may comprise different areas of the brain for example. Such tissue imaging information may comprise a Magnetic Resonance Image (MRI), a Computed Tomography (CT) image or other type of image. Often, one or more images, such as an MRI, CT, and/or a brain atlas are scaled and combined in a single image model. As one skilled in the art will understand, the location of the lead(s) can be precisely referenced to the tissue structures 114i because the lead(s) are implanted using a stereotactic frame (not shown). This allows the clinician programmer 70 on which GUI 100 is rendered to overlay the lead image 111 and the electric field image 112 with the tissue imaging information in the visualization interface 106 so that the position of the electric field 112 relative to the various tissue structures 114i can be visualized. The image of the patient's tissue may also be taken after implantation of the lead(s), or tissue imaging information may comprise a generic image pulled from a library which is not specific to the patient in question.

The various images shown in the visualization interface 106 (i.e., the lead image 111, the electric field image 112, and the tissue structures 114i) can be three-dimensional in nature, and hence may be rendered in the visualization interface 106 in a manner to allow such three-dimensionality to be better appreciated by the user, such as by shading or coloring the images, etc. Additionally, a view adjustment interface 107 may allow the user to move or rotate the images, using cursor 101 for example.

GUI 100 can further include a cross-section interface 108 to allow the various images to be seen in a two-dimensional cross section. Specifically, cross-section interface 108 shows a particular cross section 109 taken perpendicularly to the lead image 111 and through split-ring electrodes E5, E6, and E7. This cross section 109 can also be shown in the visualization interface 106, and the view adjustment interface 107 can include controls to allow the user to specify the plane of the cross section 109 (e.g., in XY, XZ, or YZ planes) and to move its location in the image. Once the location and orientation of the cross section 109 is defined, the cross-section interface 108 can show additional details. For example, the electric field image 112 can show equipotential lines allowing the user to get a sense of the strength and reach of the electric field at different locations. Although GUI 100 includes stimulation definition (102, 104) and imaging (108, 106) in a single screen of the GUI, these aspects can also be separated as part of the GUI 100 and made accessible through various menu selections, etc.

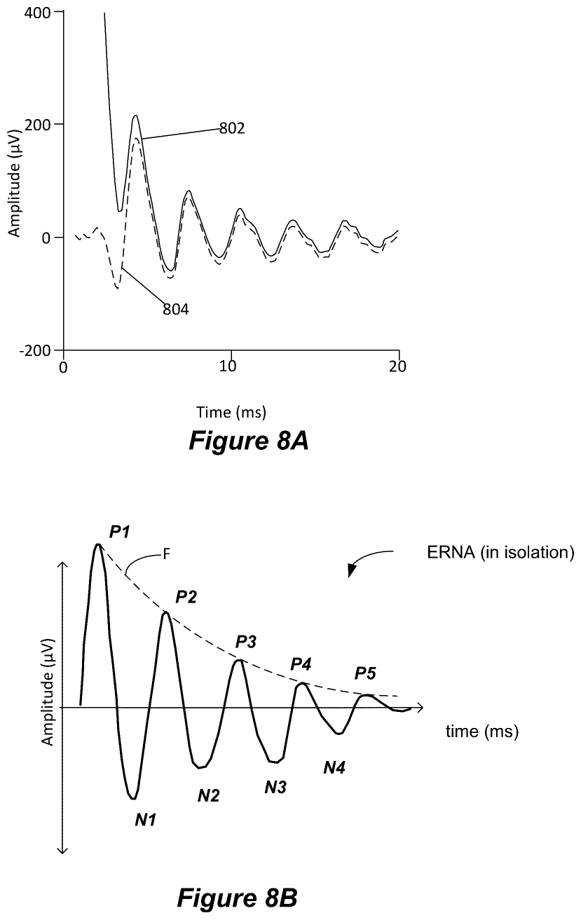

It has been observed that DBS stimulation in certain positions in the brain can evoke neural responses, i.e., electrical activity from neural elements, which may be measured. One example of such neural responses are resonant neural responses, referred to herein as evoked resonant neural activity (ERNAs). See, e.g., Sinclair, et al., “Subthalamic Nucleus Deep Brain Stimulation Evokes Resonant Neural Activity,” Ann. Neurol. 83(5), 1027-31, 2018. The ERNA responses typically have an oscillation frequency of about 200 to about 500 Hz. Stimulation of the STN, and particularly of the dorsal subregion of the STN, has been observed to evoke strong ERNA responses, whereas stimulation of the posterior subthalamic area (PSA) does not evoke such responses. Thus, ERNA can provide a biomarker for electrode location, which can potentially indicate acceptable or perhaps optimal lead placement and/or stimulation field placement for achieving the desired therapeutic response. FIG. 8A illustrates an example of an ERNA epoch after filtering 802 and after down-sampling 804 and removal of the residual stimulation artifact. FIG. 8B illustrates an example of an idealized ERNA in isolation. The ERNA comprises several positive peaks Pₙ and negative peaks Nₙ, which may have one or more characteristic amplitudes, lengths, separations, latencies, or other features. The ERNA signal may decay according to a characteristic decay function F. Such oscillatory evoked neural responses may also be referred to as DBS Local Evoked Potentials (DLEPs) and/or Evoked Oscillating Neural Responses (EONRs). The term ERNA will be used in this disclosure to refer to oscillatory evoked neural potentials in the patient's brain, such as those illustrated in FIGS. 8A and 8B. It will be appreciated that the term ERNA refers to such signals, whether or not the signals are “resonant” in the strictest mathematical or physiological sense. More generally, evoked neural responses in the patient's brain may simply be referred to as evoked neural responses.

This disclosure particularly relates to methods, workflows, and systems for using recorded neural activity, specifically ERNA, as a biomarker to inform aspects of neuromodulation therapy, such as DBS therapy. According to embodiments of the disclosure, recorded/sensed ERNA signals can provide a biomarker indicative that the appropriate neural structures for addressing the patient's symptoms are being stimulated. The stimulation and exhaustion of those neural structures and the propagation of the effects of stimulation through the neural network correlate to changes, and ideally improvements, in the patient's symptoms. As the target neural structures are stimulated and exhausted, features of the sensed ERNA signals change over time. Accordingly, an aspect of the disclosure involves monitoring temporal progression of features of the ERNA signals recorded during stimulation. The temporal progression of the ERNA signals provides a biomarker that the stimulation is affecting the correct target structures for treating the patient's symptoms. The changes in ERNA may be correlated with changes in the patient's disease state over time.

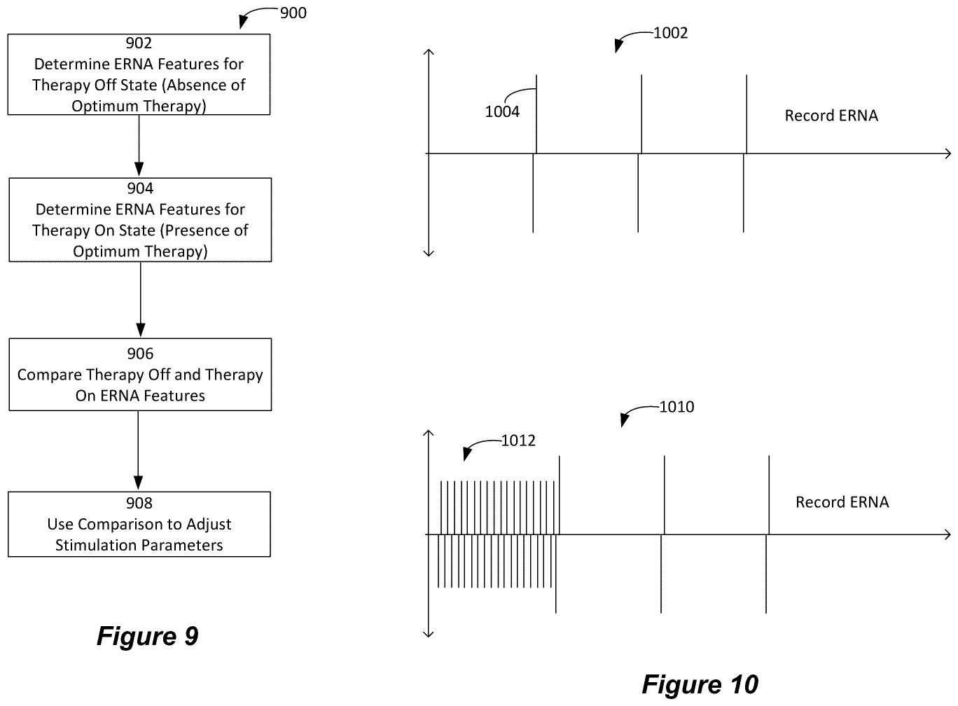

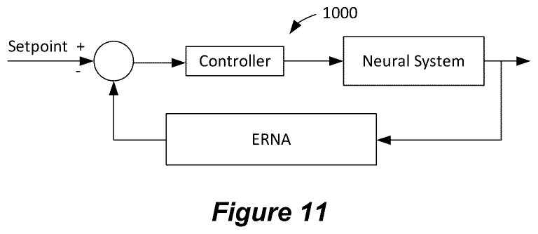

FIG. 9 illustrates an embodiment of a workflow 900 according to aspects of the disclosure. In the context of the workflow 900, it is assumed that a patient has undergone an implantation procedure whereby one or more electrode leads have been implanted in appropriate structures of the patient's brain. For example, the electrode lead(s) may be configured like the electrode lead 33 (FIG. 1B). One or more of the electrodes of the electrode lead may be configured for providing simulation to the patient's brain and/or sensing neural potentials within the patient's brain. Steps 902 and 904 involve determining features of ERNA signals recorded in a therapy-off state and a therapy-on state of stimulating the patient, respectively. As used herein, the term “therapy-off” state refers to a state of the patient wherein the patient is more strongly exhibiting symptoms of their disease, for example, tremor, etc. The therapy-off state may be when the patient is not receiving any therapeutic treatment, or it may be when the therapeutic treatment the patient is receiving is not effective at treating the patient's symptoms. As used herein, the term “therapy-on” state refers to a state of the patient wherein the stimulation is acutely treating the patient's symptoms and the patient is having a positive impact on the patient's symptoms. Ideally, the therapy-on state provides the best treatment that the patient and the clinician can expect under a given set of conditions. Aspects of the disclosure are directed to determining and maintaining stimulation parameters that provide the best therapy-on state.

Step 902 involves determining one or more features of ERNA evoked in the therapy-off state (i.e., when the patient is not receiving optimum therapy). One or more of the electrodes of the implanted electrode lead(s) may be configured to provide evoking stimulation to the patient's brain to evoke ERNA, which may be recorded at one or more of the electrodes. As used herein, the term “evoking stimulation” and/or “non-therapeutic stimulation” refers to stimulation that is applied to the patient's brain and is configured for the purposes of evoking an ERNA response and not specifically for the purpose of acutely treating the patient's symptoms. The term “therapeutic stimulation” refers to stimulation that is applied to the patient's brain and is configured to treat the patient's symptoms. The evoking stimulation and the therapeutic stimulation may comprise different stimulation parameters from each other. The therapeutic stimulation might give rise to detectable ERNA, but the therapeutic stimulation's purpose is to treat the patient's symptoms and the therapeutic stimulation is configured as such. Likewise, the evoking stimulation (i.e., the “non-therapeutic stimulation”) may incidentally have a measurable positive impact on the patient's symptoms, but its purpose is to evoke ERNA, not to acutely treat the patient's symptoms.

FIG. 10 illustrates an embodiment of an evoking stimulation waveform 1002 configured for evoking ERNA in the therapy-off state. The illustrated stimulation waveform 1002 comprises high amplitude pulses 1004 at low frequency (e.g., 10 pulses delivered at about 5-50 Hz). The ERNA signals may be recorded between the pulses 1004 and/or following a series of such pulses, and one or more features of the ERNA signal may be extracted from the signal to characterize the signal. Examples of such features of the evoked potentials include but are not limited to:
a height of any peak (e.g., N1);
a peak-to-peak height between any two peaks (such as from N1 to P2);
a ratio of peak heights (e.g., N1/P2);
a peak width of any peak (e.g., the full-width half-maximum of N1);
an area or energy under any peak;
a total area or energy comprising the area or energy under positive peaks with the area or energy under negative peaks subtracted or added;
other measures of energy of magnitude of a peak or peaks, such as an RMS measure;
a length of any portion of the curve of the evoked potential (e.g., the length of the curve from P1 to N2, calculated by various methods, including piecewise sum);
any time defining the duration of at least a portion of the evoked potential (e.g., the time from P1 to N2);
latencies of any peaks (P1 . . . Pn, N1 . . . Nn, etc.) as well as other feature-to-feature latencies;
amplitude decay function;
a time delay from stimulation to issuance of the evoked potential, which is indicative of the neural conduction speed of the evoked potential, which can be different in different types of neural tissues, such delays optionally calculated from rising edges, falling edges, or at pre-determined positions within a pulse width, such positions variable and programmable, and including determination based in whole or part from prior data from same or different patients, or computational models;
a conduction speed (i.e., conduction velocity) of the evoked potential, which can be determined by sensing the evoked potential as it moves past different sensing electrodes;
a measure of variation of any of the previous or other features, e.g., variance or standard deviation;
a rate of variation of any of the previous features, i.e., how such features change over time;
parameters fit to models of rates of changes of features, e.g., envelope, dwell-time, decay constant;
a power (or energy) determined in a specified frequency band (e.g., delta, alpha, beta, gamma, etc.) determined in a specified time window (for example, a time window that overlaps the neural response, the stimulation artifact, etc.);
spectral characteristics in the frequency domain (e.g., Fourier transform);
a cross-correlation or cross-coherence of the evoked potential shape with a target optimal shape;
a nonlinear transform applied to the signal, such as a neural network; and
any mathematical combination or function of these features.
   ERNA features that are particularly relevant in embodiments of the disclosure are the latency of the P1 peak and the ERNA amplitude (e.g., the amplitude of the P1 peak or the P1−N1 difference).

Referring again to FIG. 9, step 904 involves recording ERNA signals when the patient is in the therapy-on state. According to some embodiments, therapeutic stimulation may be provided to the patient for a period of time. For example, the therapeutic stimulation may comprise higher frequency stimulation, as is known in the art for therapeutic DBS stimulation. For example, the therapeutic stimulation may have a frequency of between 100 Hz and 150 Hz, for example, about 130 Hz, as is common in the art. Generally, the therapeutic stimulation may comprise any waveform parameters that are determined to be effective at improving the patient's symptoms. According to some embodiments, the ERNA evoked by the therapeutic stimulation may be recorded while the therapeutic stimulation is being provided. According to other embodiments, the therapeutic stimulation may be stopped, and then evoking stimulation may be provided to evoke ERNA signals. The evoking/non-therapeutic stimulation may have a lower frequency than the therapeutic stimulation. For example, the evoking/non-therapeutic stimulation may have a frequency of about 5 Hz to about 50 Hz, for example, a frequency of about 10 Hz. FIG. 10 illustrates a waveform 1010, configured for recording ERNA signals when the patient is in the therapy-on state. The waveform comprises a duration of therapeutic stimulation 1012. The therapeutic stimulation is followed by evoking stimulation having a lower frequency and a higher amplitude, during which ERNA is recorded (e.g., 10 pulses delivered at 10 Hz).

Once ERNA from the therapy-off and therapy-on states are recorded, features may be extracted from the signals and compared (FIG. 9, step 906). According to some embodiments, it is expected that the amplitude of the ERNA signal will decrease during the therapy-on state. According to some embodiments, it is expected that the latency of the ERNA (e.g., the time between the evoking stimulation and the arrival of the P1 peak) will increase during the therapy-on state. According to some embodiments, a series of ERNA measurements may be taken over a duration after therapeutic stimulation has ceased. It is expected that the ERNA features will revert to the therapy-off state over time after therapeutic stimulation has ceased. Observing the progression of the signals back to the therapy-off behavior can serve as confirmation that the recorded signals are indeed ERNA and not some other neural behavior. Likewise, a shift in ERNA features (between therapy-off and therapy-on states) may depend on the frequency of stimulation. Such frequency dependence can be indicative that the recorded signal is indeed ERNA.

According to some embodiments, the progression of the ERNA features from the therapy-off behavior to the therapy-on behavior can be correlated with improvements in the patient's symptoms when stimulation is provided. For example, various stimulation parameter sets can be provided to the patient and the patient's symptom improvement with each of the parameter sets can be observed, along with the progression to the therapy-on behavior of the ERNA recorded for each of the parameter sets. The therapy-on behavior of the ERNA signal (e.g., the ERNA amplitude and/or latency) for the best parameter set(s) can be flagged as being indicative of optimum therapy, i.e., therapy that is best stimulating and/or exhausting the appropriate excitatory neural elements implicated in the patient's disease state. The identification of therapy-on ERNA behavior can be used to facilitate and maintain the adjustment of stimulation parameters (FIG. 9, step 908). Examples of such stimulation parameters include aspects of the stimulation waveform, such as amplitude, frequency, pulse width, duty cycle, etc. The stimulation parameter adjustment may also include adjusting the electrode configuration, i.e., the selection of which one or more electrodes are active for delivering the stimulation and how much current is fractionated to each active electrode.

Aspects of the disclosure relate to analysis algorithms that leverage the progression of ERNA during therapy-off and therapy-on states to inform the adjustment of stimulation parameters. Such algorithms may be executed using control circuitry of a computing device, such as the CP 70 (FIG. 5) described above. According to some embodiments, discrimination techniques, such as machine learning and/or simple correlations may be used to identify threshold values that define what ERNA feature characteristics correlate to optimum/non-optimum therapeutic stimulation. For example, according to some embodiments, optimum therapy may be maintained when the ERNA peak width is maintained within a pre-defined range or with respect to a pre-defined threshold value. Likewise, the P1 latency may be maintained within a range or with respect to a predefined threshold. According to some embodiments, a ratio of two or more ERNA feature values may be linked, for example, within one as a baseline measurement or a measurement taken when the patient is showing no symptoms.

Some embodiments may use template ERNA signals corresponding to ideal therapy-off and/or therapy-on states. According to some embodiments, a template that represents a full ERNA recording representing a disease state (therapy-off) and an attenuated ERNA recording indicative of a better state (therapy-on) may be preloaded. Then, ERNA signals recorded during the provision of stimulation may be overlaid, correlated, and/or convolved with the template. The value of the correlation or convolution may represent how close or how far a patient is to a preferred (therapy-on) or not (therapy-off) state. Such analysis may be in the frequency domain and/or in the time domain.

The methods described above for discriminating between the behavior of ERNA features in the therapy-off and therapy-on states can be used during a clinical fitting session to help the clinician determine stimulation parameters (amplitude, pulse width, frequency, duty cycle, electrode configuration, etc.) that are best for treating the patient's symptoms, i.e., stimulation parameters that effectively modulate the neural elements associated with the patient's disease. For example, during a fitting session, evoking stimulation can be used to determine ERNA responses in the therapy-off state. Such ERNA characteristics may be mapped against available data regarding disease severity and stimulation and/or medication may be recommended accordingly. Therapeutic stimulation may be provided and ERNA progression from the therapy-off state to the therapy-on state can be observed. For example, a decay in the ERNA amplitude may be observed. Then the duty cycle of the stimulation may be ramped down, for example, to a duty cycle of 90%, then to 80%, etc., until the ERNA amplitude increases to above a predetermined threshold (indicating a reversion of the ERNA back toward the therapy-off state). Such ERNA progression may be used to determine a minimum duty cycle that maintains the ERNA behavior within a threshold corresponding to the therapy-on state. Such ERNA-guided programming may be combined with adjustments based on observation of the patient's symptoms during programming. Similar adjustments may be made to other stimulation parameters, such as the amplitude and/or pulse width, to determine stimulation parameters that maintain the therapy-on ERNA behavior.

The progression of ERNA features between the therapy-off and therapy-on states can also be used to maintain chronic therapy, for example, using one or more closed loop feedback control algorithms. According to some embodiments, such closed loop feedback control algorithms may be configured within memory and/or the control circuitry of the patient's IPG (see, e.g., 140, FIG. 6). According to some embodiments, the IPG may be configured with a diagnostic schedule feature, whereby the IPG will periodically default into a diagnostic mode. The diagnostic mode may be implemented, for example, on a monthly or a weekly basis. In the diagnostic mode the IPG may switch from providing chronic therapeutic stimulation to providing evoking stimulation. The recorded ERNA features may be compared to the therapy-on and therapy-off ERNA features described above, which may be stored in memory of the IPG. An adjustment algorithm may be used to adjust the stimulation parameters to attempt to obtain ERNA features commensurate with optimum therapy-on features determined during the fitting procedure, as described above. If the system is not able to recover such optimum therapy-on features, then the patient may be prompted to visit a clinician. According to some embodiments, the patient's external controller (60, FIG. 5) may be used to prompt the patient to seek further clinical assistance. According to some embodiments, evoking stimulation may be issued to determine new baseline therapy-off values.

According to some embodiments, the IPG may be configured for continuous monitoring of recorded ERNA features for closed loop feedback control. The IPG may be configured to record ERNA signals continuously (or semi-continuously) while chronic therapeutic stimulation is provided. One or more features of ERNA, such as the P1 and/or N1 peak may be monitored and tracked. A shift in the monitored feature(s) may indicate a change in the effectiveness of the stimulation therapy. According to some embodiments, the monitored features may be recorded and tracked to track signal and disease state stability. According to some embodiments, a shift relative to a threshold value and/or a correlation analysis may cause an alarm or flag may be instantiated. The patient may be alerted (e.g., via their external controller), asked about their symptoms, and if appropriate, prompted to seek a clinical visit. According to some embodiments, the continuous monitoring via recorded ERNA signals may be combined with data from one or more sensors, such as wearable sensors, that are configured to monitor aspects of the patient's symptoms. For example, a wearable accelerometer may be used to monitor the patient's tremor. Such external data may be used to validate the ERNA measurements and/or to toggle sensing when symptoms change. FIG. 10 illustrates a simplified closed loop feedback control algorithm 1000, whereby a controller seeks to control stimulation to maintain one or more ERNA features with respect to a set point. The control algorithm 1000 may be embodied and executed in the control circuitry of the IPG, for example. The set point may be a value, range, or threshold value for one or more ERNA features described above that correspond to stimulation providing a therapeutic benefit. Interrupts may be issued based on a comparison of the one or more neural features with the setpoint. The interrupts may trigger calling or switching between pre-defined stimulation programs. Alternatively, the control scheme may involve controllers such PID controllers, Kalman filters, or the like, which may be configured to adjust one or more stimulation parameters, such as the stimulation current (or voltage) amplitude, pulse width, frequency, or the like.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for providing deep brain stimulation (DBS) to a patient's brain using one or more electrode leads implanted in the patient's brain, wherein each of the one or more electrode leads comprises a plurality of electrodes configured to contact the patient's brain tissue, the method comprising:
using one or more of the electrodes to provide non-therapeutic stimulation to the patient's brain, wherein the non-therapeutic stimulation is configured to evoke first evoked neural resonant activity (ERNA) in the patient's brain and is not configured to treat the patient's symptoms,
using one or more of the electrodes to record first signals in the patient's brain indicative of the first ERNA, wherein the first ERNA is indicative of the patient's brain state in the absence of therapeutic stimulation,
using one of more of the electrodes to provide therapeutic stimulation to the patient's brain, wherein the therapeutic stimulation is configured to treat the patient's symptoms,
using one or more of the electrodes to record second signals indicative of second ERNA, wherein the second ERNA is indicative of the patient's brain state in the presence of therapeutic stimulation,
comparing the first ERNA and the second ERNA, and
configuring the therapeutic stimulation based on the comparison.

2. The method of claim 1, wherein the non-therapeutic stimulation has a frequency of about 5 to about 50 Hz.

3. The method of claim 1, wherein the therapeutic stimulation has a frequency of about 100 to about 150 Hz.

4. The method of claim 1, wherein comparing the first and second ERNA comprises extracting one or more features from the first and second signals.

5. The method of claim 4, wherein the one or more features comprise one or more of a latency of one or more peaks and an amplitude of one or more peaks.

6. The method of claim 4, wherein comparing the first and second ERNA comprises measuring a difference between the one or more extracted features of the first signal and the one or more extracted features of the second signal.

7. The method of claim 4, wherein comparing the first and second ERNA comprises determining a ratio of the one or more extracted features of the first signal and the one or more extracted features of the second signal.

8. The method of claim 1, wherein the second signals are recorded while the therapeutic stimulation is provided to the patient's brain.

9. The method of claim 1, wherein recording the second signals comprises:
providing the therapeutic stimulation for a first duration,
ceasing the therapeutic stimulation and providing the non-therapeutic stimulation, and
recording the second signals in response to the non-therapeutic stimulation.

10. The method of claim 9, further comprising providing the non-therapeutic stimulation for a second duration and recording a progression of the ERNA during the second duration.

11. The method of claim 1, wherein configuring the therapeutic stimulation comprises adjusting the stimulation to maintain a feature of the second ERNA with respect to a threshold value and/or a range of values.

12. The method of claim 11, further comprising issuing an alert if the feature of the second ERNA is outside the threshold and/or range of values.

13. The method of claim 1, wherein configuring the therapeutic stimulation comprises adjusting an amplitude of the stimulation.

14. The method of claim 1, wherein configuring the therapeutic stimulation comprises determining an optimized electrode configuration for providing the therapeutic stimulation, wherein the optimized electrode configuration comprises optimized one or more electrodes used to deliver the therapeutic stimulation.

15. The method of claim 14, wherein determining an optimized electrode configuration comprises:
(i) applying the non-therapeutic stimulation and the therapeutic stimulation using a trial electrode configuration comprising a trial one or more electrodes for delivering the stimulation,
(ii) determining if one or more features of the first and second ERNAs differ by greater than a predetermined threshold value,
(iii) if the one or more of the features differ by greater than the predetermined threshold value, using the trial electrode configuration as the optimized electrode configuration for the therapeutic stimulation, and
(iv) if the one or more of the features do not differ by greater than the predetermined threshold value, iteratively repeating steps (i-iii) with different trial electrode configurations until the one or more of the features differ by greater than the predetermined threshold value for that trial electrode configuration and using that trial electrode configuration as the optimized electrode configuration for the therapeutic stimulation.

16. The method of claim 15, wherein the one or more features comprise one or more of a latency of one or more peaks and an amplitude of one or more peaks.

17. The method of claim 1, further comprising tracking one or more values of one or more features of the first ERNA over time.

18. The method of claim 17, further comprising determining one or more trends of the one or more values of one or more features of the first ERNA over time.

19. The method of claim 18, wherein the one or more trends are indicative of a progression or an improvement in the patient's disease state.

20. The method of claim 18, further comprising adjusting the stimulation based on the one or more trends.
